(19) United States
(12) Patent Application Publication
(10) Pub. No.: US 2010/0130830 A1
(43) Pub. Date: May 27, 2010

(54) Foot examination and measurement system and method of the same

(76) Inventor: Wei-Cheng Lin, Luodong Town (TW)

Correspondence Address: SCHMEISER, OLSEN & WATTS, 22 CENTURY HILL DRIVE, SUITE 302, LATHAM, NY, 12110, US

(21) Appl. No.: 12/313,858

(22) Filed: Nov. 24, 2008

(51) Int. Cl. A61B 5/00

(52) U.S. Cl. 600/300; 705/3

(57) ABSTRACT

The present invention relates to a foot examination and measurement system and method of the same. A scanning device, a storage device, a computing device, and an interface device are provided to examine and measure a user's foot to get various information about the user's foot, such as foot pressure distribution, foot length, width and, angle, etc. The system and method can further include a databank for measuring positions of different areas on the user's sole; or a first image capturing device for measuring the user's heel angle; or a second image capturing device for measuring the user's dynamic foot pressure distribution when the user walks. With these functions, the invention is able to assist the user in choosing shoes and/or insoles most suitable for the user's feet in order to improve the user's foot and whole body health.

### FIELD OF THE INVENTION

The present invention relates to a foot examination and measurement system and method of the same for diagnostic purpose, and more particularly to a system for examining, measuring and recording the shapes, modes, sizes, and movements of a whole of or parts of a human body.

### BACKGROUND OF THE INVENTION

Human being's feet are the most precise and complicated structure among the masterpieces of God's art. The feet occupies one fourth of the bones on the human body, and includes tightly connected and arranged bones, muscles, ligaments and joints. When this complicated structure has problems, different parts of the human body, including knees, pelvis, spine, or even different internal organs, would be disadvantageously affected to result in an unhealthy body as a house having an inclined foundation. Therefore, it is very important for one to maintain two healthy feet to ensure the whole body health.

However, foot problems are quite often found among people. According to a statistical material from the United States, the number of women having foot problems is four times as high as the number of men having foot problems. A result from an investigation by questionnaire indicates 87% of the people in the investigation have or had ached foot. And, 25% of the 1.6 millions of diabetic foot patients in the United States are subject to the danger of amputation.

According to investigations conducted in Japan, there are about 55 to 85% Japanese have had tired and uncomfortable feet, and about 50% of Japanese have had sore and painful waist, back, and hip joints. Moreover, almost 100% of pregnant women have had uncomfortable feet, waist, and back.

In Taiwan, about 60% of people say that they have had the symptom of painful feet. From investigations conducted in Taiwan, most people do not think they have ever worn correct shoes. About 55% of Taiwanese feel that their shoes are not comfortable enough for wearing, and about 26% feel that the shoes do not fit their feet. According to statistic materials from studies on flat foot in Taiwan, 13% residents in Kaohsiung, 23% residents in Taichung, 40% residents in Tainan, 25% and 31% residents in I-Lan in 2001 and 2002, respectively, are suffering from flat foot. In other words, in Taiwan, a large number of children in the stage of development are suffering from flat foot due to the use of a baby walker at a too much early stage of development to result in overloaded feet of children. Therefore, the occurrence rate of flat foot in Taiwan has now become higher because of the acquired flat foot.

Currently, a conventional inked footprint is used to determine a person's foot pressure distribution. The inked footprint is obtained by applying an amount of ink over a footprint imprint kit and causing the person to step on the imprint kit, so that the person's footprint is transferred to a white paper. The application of the ink over the imprint kit and the analysis of the footprint all require an experienced operator, so as to correctly determine the person's foot pressure distribution and whether the person has any foot disease. Since the footprint indirectly produced in the above-described conventional way is only visually observed with unaided eyes, there is a great possibility the analysis and determination about the footprint is incorrect or inaccurate.

Also a foot pressure sensing device is available in the market. This foot pressure sending device includes a plurality of pressure sensors. When a person steps on the foot pressure sensing device, the plurality of pressure sensors would immediately detect pressure values appeared in different small areas on the person's sole. The detected pressure values are then processed to produce a picture showing the pressure distributed over the person's whole foot. The above-mentioned foot pressure sensing device provides considerably accurate measurement and measuring results. However, the sensing device includes a large number of pressure sensors, which increase not only the overall cost, but also the future maintenance cost of the sensing device. As a result, the foot pressure sensing device could not become widely acceptable in the market.

Meanwhile, in selling shoes, a senior salesclerk determines a customer's shoe size by eye estimation and asking the customer to try on the shoes. And, in hand-making customized insoles, the maker determines the arch shapes and lengths of different sections of the customer's feet by directly touching the customer's feet or using the above-described inked footprints, and modifies the insoles through trial wearing by the customer until absolutely correct insoles are made. These steps are complicated and troublesome for both the customer and the maker.

Therefore, it is desirable to develop a foot examination and measurement system and method that provides a simple technique for examining and measuring foot, uses general image conversion principle to convert simple scanned picture and image into a simulated foot pressure distribution picture, and computes the size, shape, and other information related to foot.

### SUMMARY OF THE INVENTION

A primary object of the present invention is to provide a foot examination and measurement system and method of the same, which is able to examine and measure a user's foot to thereby provide various foot-related information, so that the user can use these information to choose shoes and/or insoles most suitable for the user's feet in order to improve the user's foot and whole body health.

To achieve the above and other objects, the foot examination and measurement system according to the present invention includes a scanning device for scanning a user's sole when the user steps on the scanning device; a storage device electrically connected to the scanning device for storing information about the user's foot outputted by the scanning device; a computing device electrically connected to the storage device for computing the user's foot information; and an interface device electrically connected to the computing device for displaying the user's foot information and computation results from the computing device.

Another object of the present invention is to provide a foot examination and measurement method for examining and measuring a user's foot pressure distribution. The method includes the following steps: providing a foot examination and measurement system including a scanning device, a storage device electrically connected to the scanning device, a computing device electrically connected to the storage device, and an interface device electrically connected to the computing device; allowing the user to step on the scanning device; using the scanning device to scan the user's sole; the scanning device outputting a full-color image and storing the same in the storage device; using the computing device to convert the full-color image into a grayscale image; using the computing device to categorize areas of different gray levels in the grayscale image, so that a pixel distribution picture consisting of a particular group of color levels is obtained; and displaying the pixel distribution picture obtained through computation on the interface device.

A further object of the present invention is to provide a foot examination and measurement method for measuring a user's actual foot length. The method includes the following steps: providing a foot examination and measurement system including a scanning device, a storage device electrically connected to the scanning device, a computing device electrically connected to the storage device, and an interface device electrically connected to the computing device; allowing the user to step on the scanning device; using the scanning device to scan the user's sole; the scanning device outputting a full-color image and storing the same in the storage device; using the computing device to mark a highest point on the full-color image at a position corresponding to a most front point on the user's second toe; using the computing device to locate a lowest point on the full-color image from bottom to top; and using the computing device to locate a centerline of the user's sole by connecting the highest point and the lowest point, calculate a distance between the highest and the lowest point to determine an actual length of the user's foot.

A still further object of the present invention is to provide a foot examination and measurement method for measuring an inclined angle of a user's first toe and fifth toe. The method includes the following steps: providing a foot examination and measurement system including a scanning device, a storage device electrically connected to the scanning device, a computing device electrically connected to the storage device, and an interface device electrically connected to the computing device; allowing the user to step on the scanning device; using the scanning device to scan the user's sole; the scanning device outputting a full-color image and storing the same in the storage device; using the computing device to compute and locate in the full-color sole image two points that correspond to the prominent points on the inner and the outer side, respectively, of the user's heel bone, and another two points that correspond to the prominent points on the inner and the outer side, respectively, of the user's forefoot; using the computing device to connect the two prominent points on the inner side of the user's foot to obtain a third extending line, and connect the two prominent points on the outer side of the user's foot to obtain a fifth extending line; using the computing device to inwardly rotate the third extending line about the prominent point on the inner side of the user's forefoot by an angle so that the third extending line touches the full-color image and thereby produces a first contacting point, and inwardly rotate the fifth extending line about the prominent point on the outer side of the user's forefoot by an angle so that the fifth extending line touches the full-color image and thereby produces a second contacting point; using the computing device to connect the prominent point on the inner side of the user's forefoot and the first contacting point to obtain a fourth extending line, and connect the prominent point on the outer side of the user's forefoot and the second contacting point to obtain a sixth extending line; and using the computing device to calculate an angle contained between the third extending line and the fourth extending line as well as an angle contained between the fifth extending line and the sixth extending line.

A still further object of the present invention is to provide a foot examination and measurement method for dividing a user's sole into different areas. The method includes the following steps: providing a foot examination and measurement system including a scanning device, a storage device electrically connected to the scanning device, a computing device electrically connected to the storage device, an interface device electrically connected to the computing device, and a databank electrically connected to the computing device; allowing the user to step on the scanning device; using the scanning device to scan the user's sole; the scanning device outputting a full-color image and storing the same in the storage device; and retrieving a plurality of data built in the databank, and the user's sole as shown in the full-color image obtained in the previous step being automatically divided into several areas according to the data retrieved from the databank.

A still further object of the present invention is to provide a foot examination and measurement method for measuring a user's foot angle. The method includes the following steps: providing a foot examination and measurement system including a scanning device, a storage device electrically connected to the scanning device, a computing device electrically connected to the storage device, an interface device electrically connected to the computing device, and a first image capturing device electrically connected to the storage device; making two marks at a point at the user's heel bottom and a point above the user's heel bone, respectively; using the first image capturing device to capture a coronal image of the user's foot at a rear side thereof and storing the captured image in the storage device; using the computing device to connect the two marks to obtain a seventh extending line; and using the computing device to compute an inclined angle between the seventh extending line and a vertical line, or another inclined angle between the seventh extending line and a horizontal line.

A still further object of the present invention is to provide a foot examination and measurement method for measuring a user's foot pressure distribution when the user steps on a scanning device like walking. The method includes the following steps: providing a foot examination and measurement system including a scanning device, a storage device electrically connected to the scanning device, a computing device electrically connected to the storage device, an interface device electrically connected to the computing device, and a second image capturing device provided in the scanning device; allowing the user to step on the scanning device like walking; using the second image capturing device to capture images of the user's soles at a first fixed rate while the user steps on the scanning device; the second image capturing device outputting a plurality of full-color images and storing the same in the storage device; using the computing device to convert the plurality of full-color images into a plurality of grayscale images; using the computing device to categorize areas of different gray levels in the grayscale images to obtain a plurality of pixel distribution pictures each consisting of a particular group of color levels; and displaying the pixel distribution pictures obtained through computation on the interface device. The method further includes a dynamic playback step of continuously playing back the plurality of pixel distribution pictures at a second fixed rate. The first and the second fixed rate can be 15 frames per second.

The foot examination and measurement system and method according to the present invention is able to examine and measure a user's foot to thereby provide various foot-related information, with which the user can choose shoes and/or insoles most suitable for the user's feet in order to improve the user's foot and whole body health.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The structure and the technical means adopted by the present invention to achieve the above and other objects can be best understood by referring to the following detailed description of the preferred embodiments and the accompanying drawings, wherein

FIGS. 1A to 1D are block diagrams of different embodiments of a system for foot examination and measurement according to the present invention;

FIG. 2 is a flowchart showing the steps according to the present invention for examining a user's foot pressure distribution;

FIG. 3 is a flowchart showing the steps included in the method for foot examination and measurement according to the present invention for converting a two-dimensional full-color image of a user's foot into a three-dimensional image of the foot;

FIG. 4 shows the calculation of a user's foot-ground contact area according to the method of the present invention for foot examination and measurement;

FIG. 5A is a flowchart showing the steps included in the method for foot examination and measurement according to the present invention for measuring a user's actual foot length;

FIG. 5B is a conceptual view showing the measurement of a user's actual foot length according to the method of the present invention;

FIG. 6A is a flowchart showing the steps included in the method for foot examination and measurement according to the present invention for measuring the width of a user's forefoot;

FIG. 6B is a conceptual view showing the measurement of the width of a user's forefoot according to the method of the present invention;

FIG. 7A is a flowchart showing the steps included in the method for foot examination and measurement according to the present invention for measuring the width of a user's middle foot;

FIG. 7B is a conceptual view showing the measurement of the width of a user's middle foot according to the method of the present invention;

FIGS. 8A and 8B is a flowchart showing the steps included in the method for foot examination and measurement according to the present invention for measuring a user's foot eversion angle;

FIG. 8C is a conceptual view showing the measurement of a user's foot eversion angle according to the method of the present invention;

FIG. 9A is a flowchart showing the steps included in the method for foot examination and measurement according to the present invention for measuring an inclined angle of a user's first toe and fifth toe;

FIG. 9B is a conceptual view showing the measurement of the inclined angle of a user's first toe and fifth toe according to the method of the present invention;

FIG. 10 is a conceptual view showing different areas defined on Asians'soles as stored in a databank included in the foot examination and measurement system of the present invention;

FIG. 11 is a flowchart showing the steps included in the method for foot examination and measurement according to the present invention for dividing a user's sole into different areas;

FIG. 12A is a flowchart showing the steps included in the method for foot examination and measurement according to the present invention for measuring a user's foot angle;

FIG. 12B is a conceptual view showing the measurement of a user's foot angle according to the method of the present invention;

FIG. 13 is a flowchart showing the steps included in the method for foot examination and measurement according to the present invention for measuring a user's foot pressure distribution when the user steps on a scanning device like walking; and

FIG. 14 is a scanned full-color image showing changes in a user's foot pressure distribution when the user steps on a scanning device like walking.

### DETAILED DESCRIPTION OF THE PREFERRED EMBODIMENTS

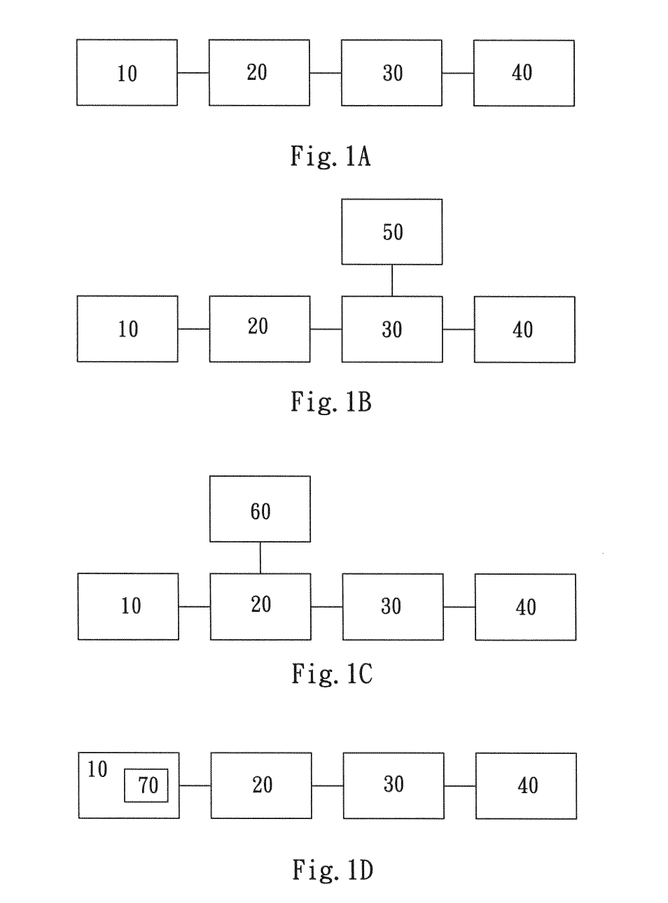

Please refer to FIG. 1A that is a block diagram of a system for foot examination and measurement according to a first embodiment of the present invention. As shown, the foot examination and measurement system in the first embodiment of the present invention includes a scanning device 10, a storage device 20, a computing device 30, and an interface device 40. The scanning device 10 is used to scan a user's foot when the user steps on the scanning device 10. The storage device 20 is electrically connected to the scanning device 10 for storing information about the user's foot outputted by the scanning device 10. The computing device 30 is electrically connected to the storage device 20 for computing the information about the user's foot. And, the interface device 40 is electrically connected to the computing device 30 for displaying the user's foot information and computation results from the computing device 30.

With the above arrangements, the foot examination and measurement system of the present invention is able to examine and measure the user's foot pressure distribution, convert a planar (two-dimensional) full-color image of the user's foot into a three-dimensional image, compute the user's foot-ground contact area, measure the user's actual foot length, measure a width of the user's forefoot, measure a width of the user's middle foot, measure the user's foot eversion angle, measure an inclined angle of the user's first toe and fifth toe, etc.

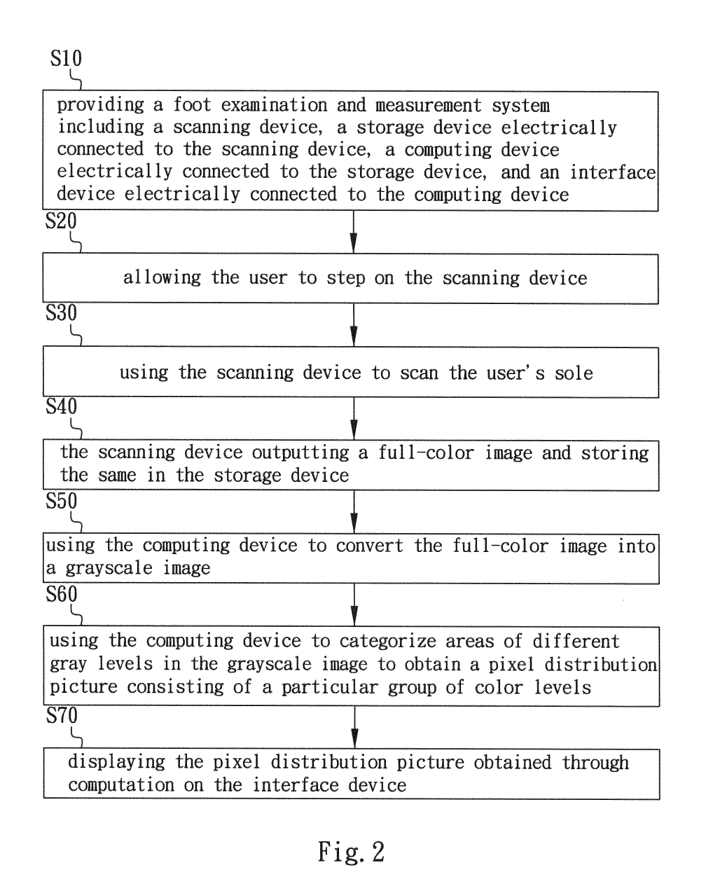

Please refer to FIG. 2 that is a flowchart showing the steps according to the present invention for examining and measuring a user's foot pressure distribution. In a first step S10, the foot examination and measurement system according to the first embodiment of the present invention is prepared. In a second step S20, the user steps on the scanning device 10. In a third step S30, use the scanning device 10 to scan the user's sole. In a fourth step S40, the scanning device 10 outputs a full-color image and stores the same in the storage device 20. In a fifth step S50, use the computing device 30 to convert the full-color image into a grayscale image. In a sixth step S60, use the computing device 30 to categorize areas of different gray levels in the grayscale image, so that a pixel distribution picture consisting of a particular group of color levels is obtained. In a seventh step S70, the pixel distribution picture obtained through computation is displayed on the interface device 40.

According to another embodiment of the present invention, in the step S50 of using the computing device 30 to convert the full-color image into a grayscale image, a further step of removing the background from the full-color image can be included.

According to an embodiment of the present invention, in the step S50 of using the computing device 30 to convert the full-color image into a grayscale image, the following conversion formula is used to perform the conversion:

Y=(9798×r+19235×g+3735×b)/32768

Where, y is a gray value, and r, g, and b are corresponding intensities of red, green, and blue colors, respectively.

According to an embodiment of the present invention, in the step S60 of using the computing device 30 to categorize areas of different gray levels in the grayscale image to obtain a pixel distribution picture consisting of a particular group of color levels, every specific range of the gray values is defined as one color level, and the gray levels in the grayscale image are sequentially converted into their respectively correspondent specific color level according to their respective gray value.

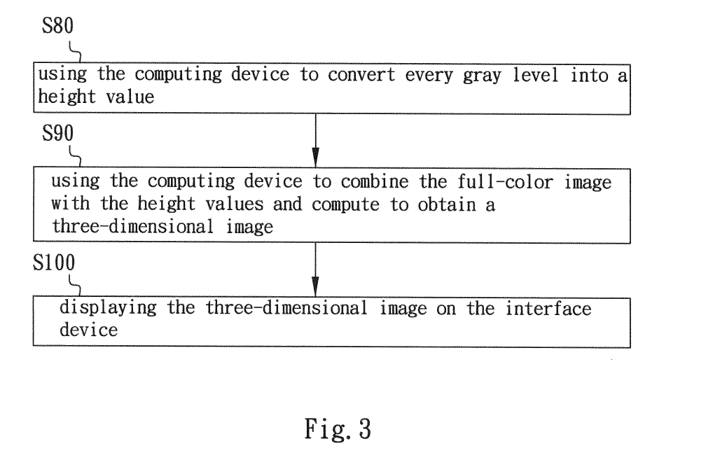

FIG. 3 is a flowchart showing the steps included in the method for foot examination and measurement according to the present invention for converting a two-dimensional full-color image of a user's foot into a three-dimensional image of the foot. To do this, in a step S80, after the above-described steps S10 to S50 are implemented to obtain the grayscale image and different gray levels and gray values for the gray scale image, the computing device 30 converts every gray level into a height value. Then, in a step S90, the computing device 30 combines the full-color image with the height values to obtain a three-dimensional image through further computation. Finally, in a step S100, the three-dimensional image is displayed on the interface device 40. With the above-described method, the pressure endured by the user's foot is presented by a three-dimensional image that can be easily understood by any non-professional person, advantageously enabling wide application of the foot examination and measurement system of the present invention and easy promotion thereof in the market.

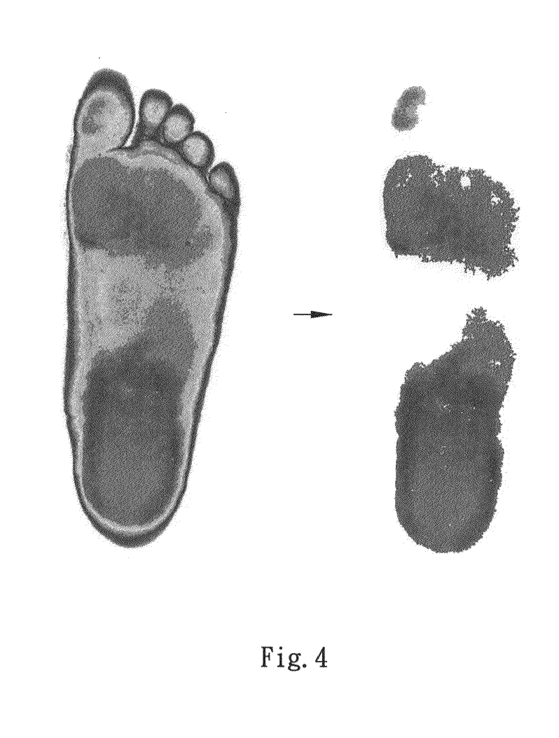

FIG. 4 shows the calculation of a user's foot-ground contact area according to the method of the present invention for foot examination and measurement. To do this, the computing device 30 divides a sum of all adjoining pixel blocks having a gray value above 144 by a total number of the pixels in the grayscale image, so as to obtain a proportion of the user's foot-ground contact area to the user's whole sole area.

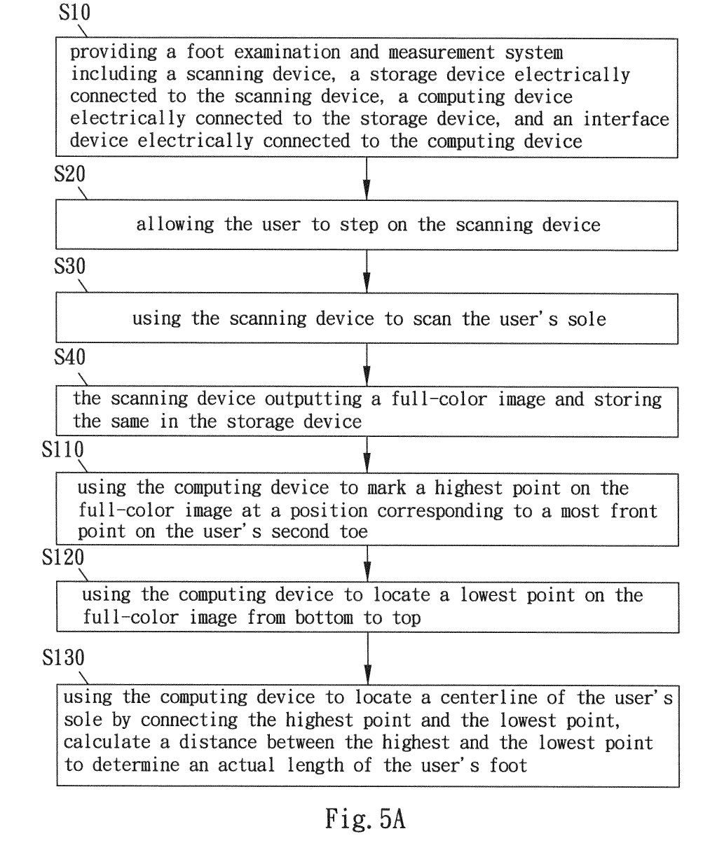

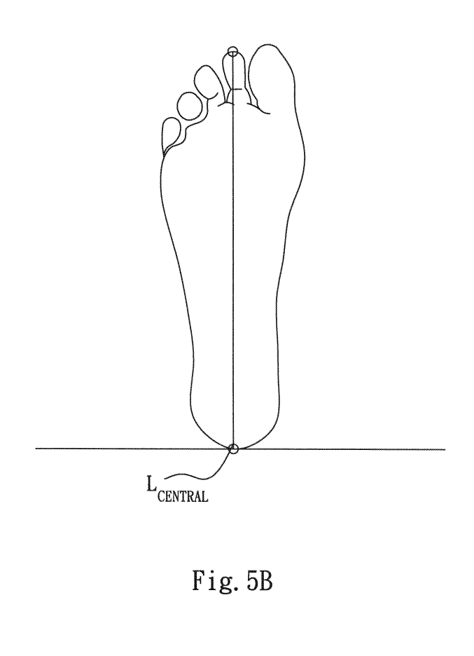

FIG. 5A is a flowchart showing the steps included in the method for foot examination and measurement according to the present invention for measuring a user's actual foot length; and FIG. 5B is a conceptual view showing the measurement of a user's actual foot length according to the method of the present invention. To do so, in a step S110 after the steps S10 to S40 are completed to obtain the full-color image of the user's sole, the computing device 30 marks a highest point on the full-color image at a position corresponding to a most front point on the user's second toe. Then in a step S120, the computing device 30 also locates a lowest point on the full-color image from bottom to top. In a step S130, the computing device 30 locates a centerline L_{CENTRAL} of the sole connecting the highest point and the lowest point, computes a distance between the highest and the lowest point, and determines an actual length of the user's foot. With the above-described method, when the user steps on the scanning device 10, no matter whether the user stands with two feet in parallel with each other or respectively directed outward at a certain angle, the actual foot length can always be accurately measured without being adversely affected by two skewed feet.

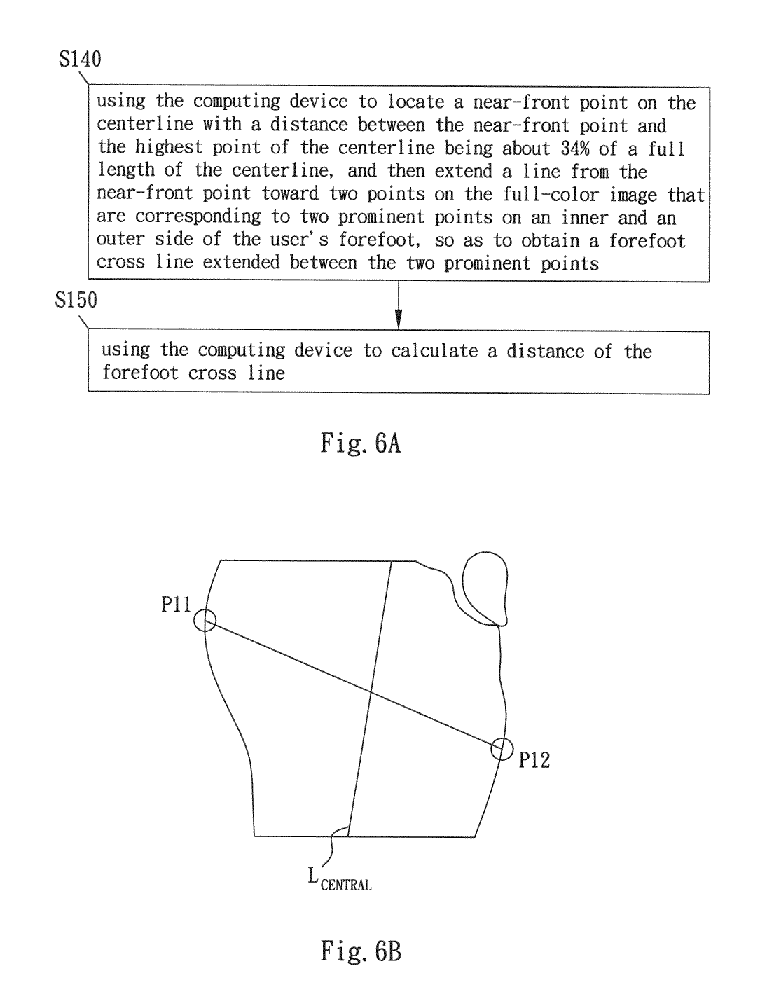

FIG. 6A is a flowchart showing the steps included in the method for foot examination and measurement according to the present invention for measuring a width of a user's forefoot; and FIG. 6B is a conceptual view showing the measurement of the width of a user's forefoot according to the method of the present invention. To do so, in a step S140 after the above steps S10 to S40 and S110 to S130 are completed to obtain the center line L_{CENTRAL}, the computing device 30 locates a near-front point on the centerline L_{CENTRAL} with a distance between the near-front point and the highest point of the centerline L_{CENTRAL} being about 34% of a full length of the centerline L_{CENTRAL}, and then, the computing device 30 extends a line from the near-front point toward two points P11, P12 on the full-color image that are corresponding to two prominent points at an inner and an outer side of the user's forefoot, so as to obtain a forefoot cross line extended between the two prominent points P11 and P12. In a step S150, the computing device 30 calculates the distance of the forefoot cross line.

In an embodiment of the present invention, the method for measuring the width of the user's forefoot further includes a manual measurement step, in which the user adjusts the positions of the two prominent points P11, P12.

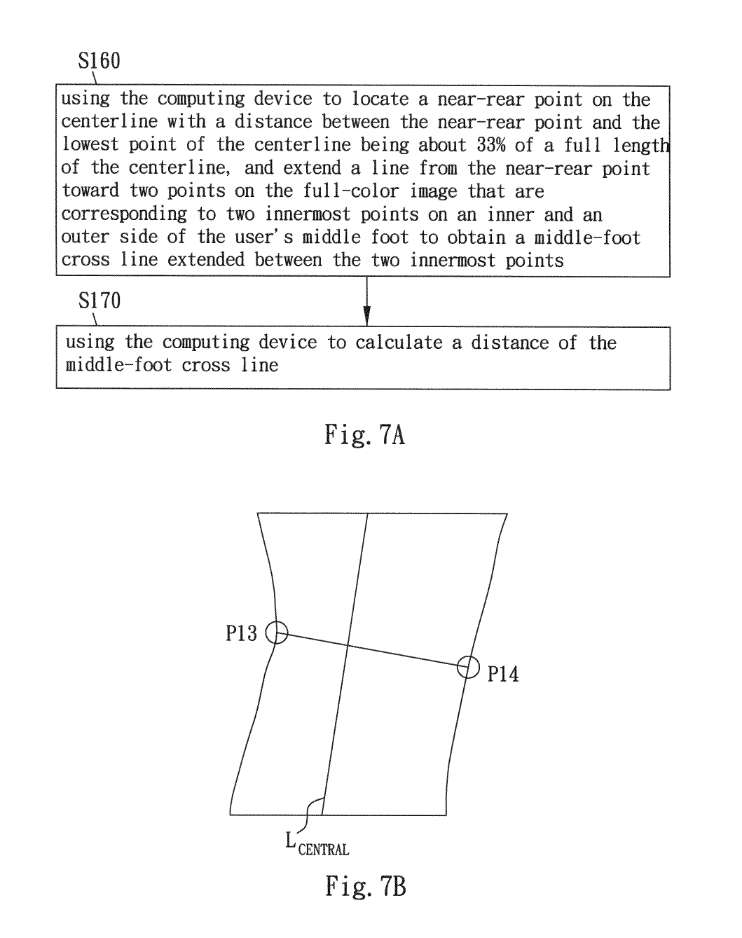

FIG. 7A is a flowchart showing the steps included in the method for foot examination and measurement according to the present invention for measuring a width of a user's middle foot; and FIG. 7B is a conceptual view showing the measurement of the width of a user's middle foot according to the method of the present invention. To do so, in a step S160 after the above steps S10 to S40 and S110 to S130 are completed to obtain the centerline L_{CENTRAL}, the computing device 30 locates a near-rear point on the centerline L_{CENTRAL} with a distance between the near-rear point and the lowest point of the centerline L_{CENTRAL} being about 33% of a full length of the centerline L_{CENTRAL}, and then, the computing device 30 extends a line from the near-rear point toward two points P13, P14 on the full-color image that are corresponding to two innermost points on an inner and an outer side of the user's middle foot, so as to obtain a middle-foot cross line extended between the two innermost points P13 and P14. In a step S170, the computing device 30 calculates the distance of the middle-foot cross line.

In an embodiment of the present invention, the method for measuring the width of the user's middle foot further includes a manual measurement step, in which the user adjusts the positions of the two innermost points P13, P14.

With the above-described method, the foot examination and measurement system of the present invention is able to automatically measure the widths of the user's forefoot and middle foot and thereby enables a non-professional person to help the user in choosing a shoe shape that is most suitable for the user. On the other hand, the foot examination and measurement method of the present invention includes the manual measurement step for a professional person to measure and obtain more accurate information about the widths of the user's forefoot and middle foot.

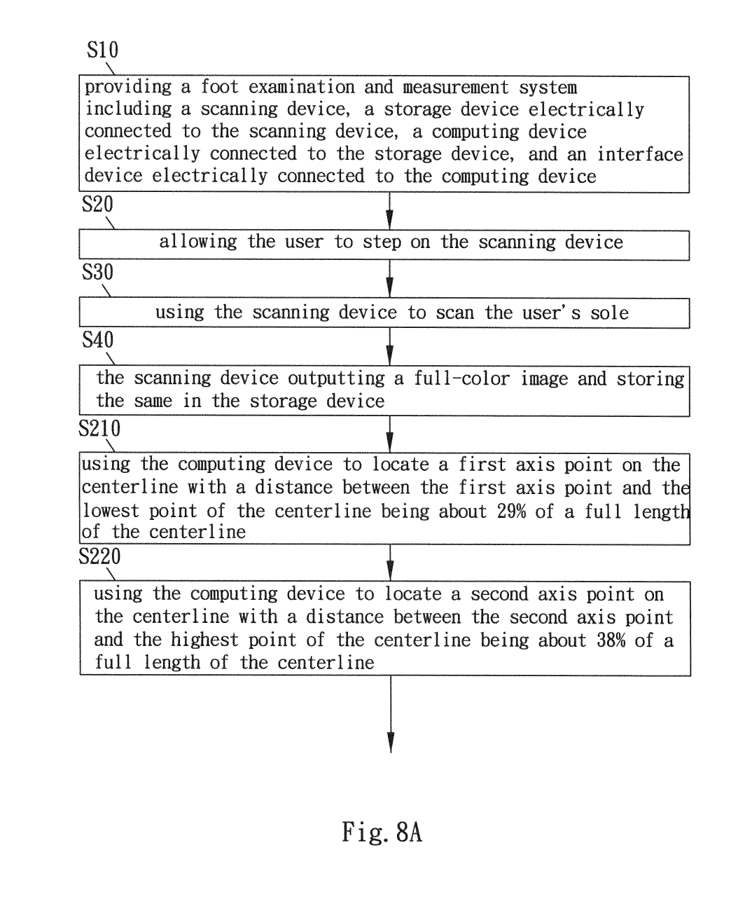

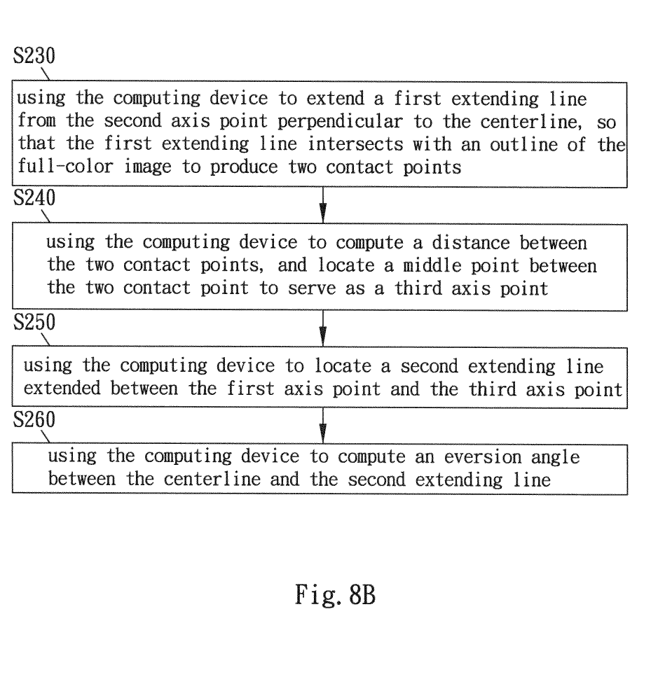

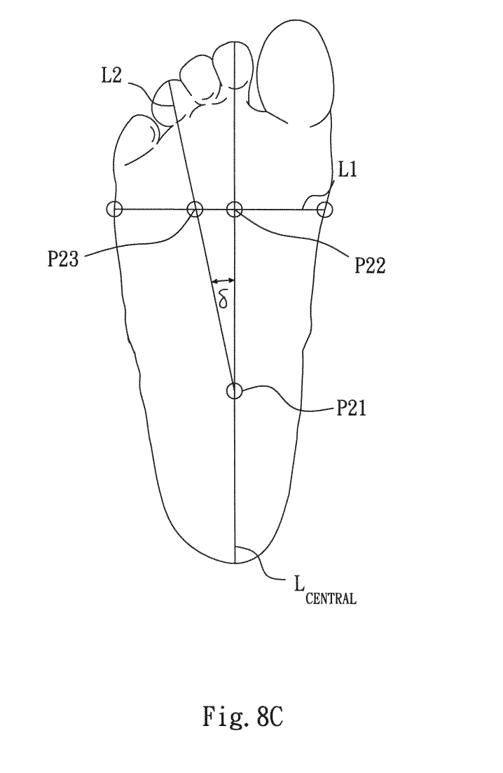

FIGS. 8A and 8B is a flowchart showing the steps included in the method for foot examination and measurement according to the present invention for measuring a user's foot eversion angle; and FIG. 8C is a conceptual view showing the measurement of a user's foot eversion angle according to the method of the present invention. To do so, in a step S210 after the steps S10 to S40 are completed to obtain the full-color image of the user's sole, the computing device 30 locates a first axis point P21 on the centerline L_{CENTRAL} with a distance between the first axis point P21 and the lowest point of the centerline L_{CENTRAL} being about 29% of the full length of the centerline L_{CENTRAL}. In a step S220, the computing device 30 locates a second axis point P22 on the centerline L_{CENTRAL} with a distance between the second axis point P22 and the highest point of the centerline L_{CENTRAL} being about 38% of the full length of the centerline L_{CENTRAL}. In a step S230, the computing device 30 extends a first extending line L1 from the second axis point P22 perpendicular to the centerline L_{CENTRAL} to intersect with an outline of the full-color image and thereby produces two contact points. In a step S240, the computing device 30 computes a distance between the two contact points, and locates a middle point between them to serve as a third axis point P23. In a step S250, the computing device 30 locates a second extending line L2 extended between the first axis point P21 and the third axis point P23. In a step S260, the computing device 30 computes an eversion angle δ between the centerline L_{CENTRAL} and the second extending line L2.

With the above-described method, the foot examination and measurement system of the present invention is able to automatically measure an eversion angle of the user's forefoot relative to the user's heel, enabling even a non-professional person to help a user in choosing a shoe shape that is most suitable for the user.

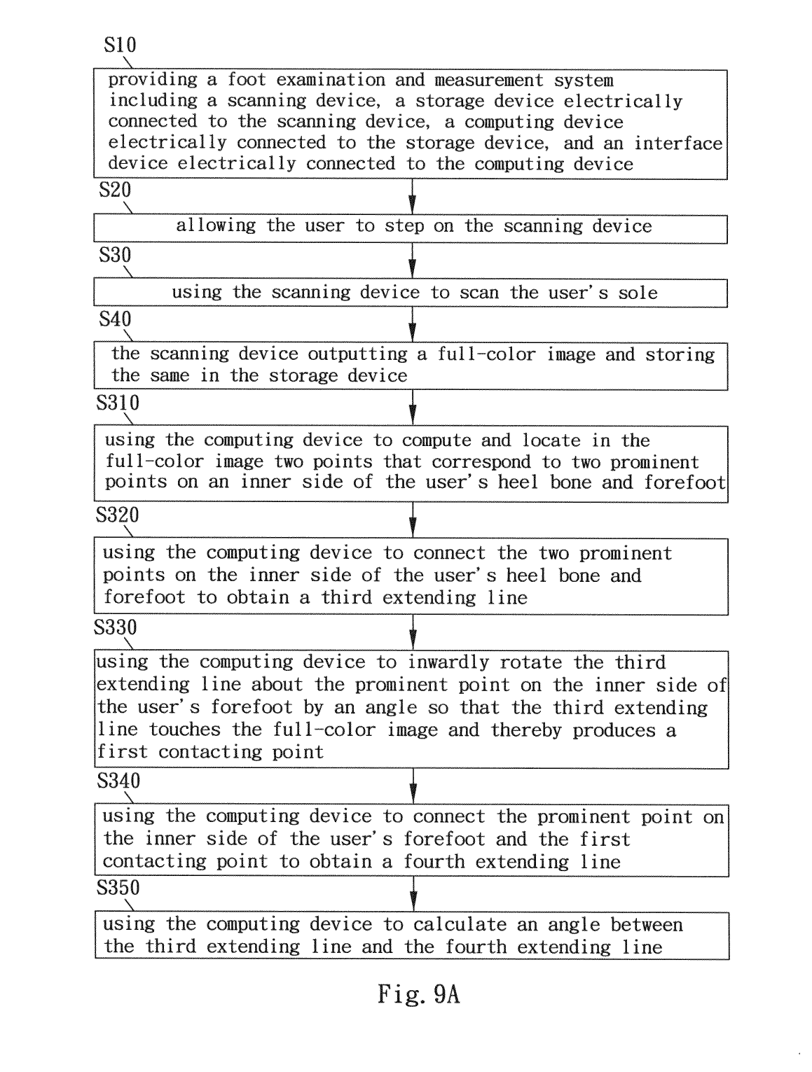

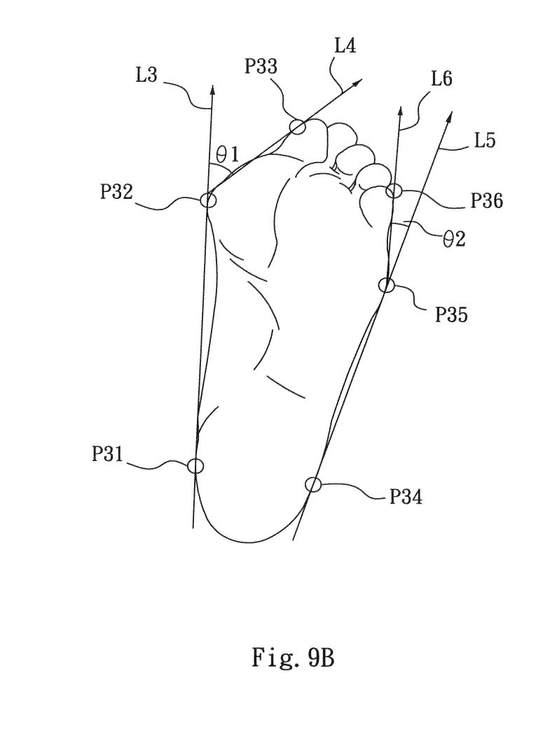

FIG. 9A is a flowchart showing the steps included in the method for foot examination and measurement according to the present invention for measuring an inclined angle of a user's first toe and fifth toe; and FIG. 9B is a conceptual view showing the measurement of the inclined angle of a user's first toe and fifth toe according to the method of the present invention. To do so, after the full-color image of the user's sole is obtained through the above-described steps S10 to S40, the computing device 30, in a step S310, computes and locates in the full-color image two points P31, P34 that correspond to two prominent points on the inner and the outer side, respectively, of the user's heel bone, and another two points P32, P35 that correspond to two prominent points on the inner and the outer side, respectively, of the user's forefoot. In a step S320, the computing device 30 connects the point P31 to the point 32 to obtain a third extending line L3, and connects the point P34 to the point P35 to obtain a fifth extending line L5. In a step S330, the computing device 30 inwardly rotates the third extending line L3 about the prominent point P32 on the inner side of the user's forefoot by an angle so that the third extending line L3 touches the full-color image and thereby produces a first contacting point P33. And, in a step S350, the computing device 30 calculates an angle θ₁ between the third extending line L3 and the fourth extending line L4 to obtain an inclined angle of a user's first toe. Similarly, the computing device 30 inwardly rotates the fifth extending line L5 about the prominent point P35 on the outer side of the user's forefoot by an angle so that the fifth extending line L5 touches the full-color image and thereby produces a second contacting point P36. In a fourth step S340, the computing device 30 connects the prominent point P32 and the first contacting point P33 to obtain a fourth extending line L4, and connects the prominent point P35 and the second contacting point P36 to obtain a sixth extending line L6. And the computing device 30 calculates an angle θ₂ between the fifth extending line L5 and the sixth extending line L6 to obtain an inclined angle of a user's fifth toe.

With the above-described method, the foot examination and measurement system of the present invention is able to automatically measure an inclined angle of the first toe and the fifth toe of the user's foot, allowing a non-professional person to help the user in choosing a shoe shape most suitable for the user.

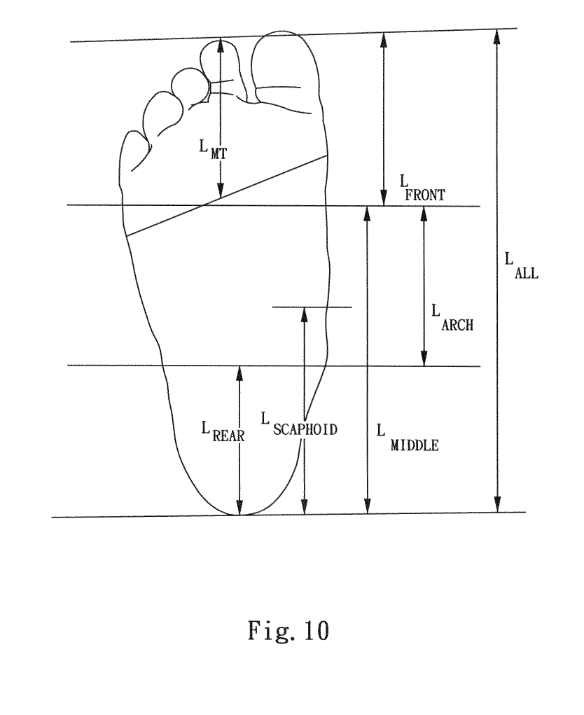

FIG. 10 is a conceptual view showing different areas defined on Asians'soles as stored in a databank included in the foot examination and measurement system of the present invention; and Table 1 listed below shows data about the sizes of different areas on Asian's soles as stored in the databank of the present invention.

TABLE 1
Data of Different Areas on Asians' Soles

| | | | Middle | | | | | |
|---|---|---|---|---|---|---|---|---|
| | Rear foot/ | Rear foot/ | foot/Whole | Front foot/ | Foot arch/ | Foot arch/ | Scaphoid/ | Scaphoid/ |
| | Whole foot | Middle foot | foot | Whole foot | Whole foot | Middle foot | Whole foot | Middle foot |
| Left foot | 0.29454 | 0.47384 | 0.62154 | 0.37846 | 0.32701 | 0.52616 | 0.405518 | 0.652406 |
| Right foot | 0.2949 | 0.47535 | 0.62079 | 0.37921 | 0.3259 | 0.52465 | 0.405393 | 0.653005 |
| Average | 0.29472 | 0.474595 | 0.621165 | 0.378835 | 0.326455 | 0.525405 | 0.4054555 | 0.6527055 |
| proportion | | | | | | | | |
| Average | 29.47% | 47.46% | 62.12% | 37.88% | 32.65% | 52.54% | 40.55% | 65.27% |
| percentage | | | | | | | | |
| Rounded | 29% | 47% | 62% | 38% | 33% | 53% | 41% | 65% |
| average | | | | | | | | |
| percentage | | | | | | | | |

| | | |
|---|---|---|
| Number of | Effective for left foot: 1229 person-time | Effective for left foot: |
| examined | | 1218 person-time |
| persons: 1400 | Effective for right foot: 1213 person-time | Effective for right foot: |
| person-time | | 1202 person-time |

Note:
(a) In the case the same person accepts two times of examination, the two examinations are conducted at an interval of longer than six months.
(b) Percentage of the forefoot joint to the whole foot is 34%
Duration of Study: 2003.01~2007.12
Study conducted by: Wei-Chan Lin

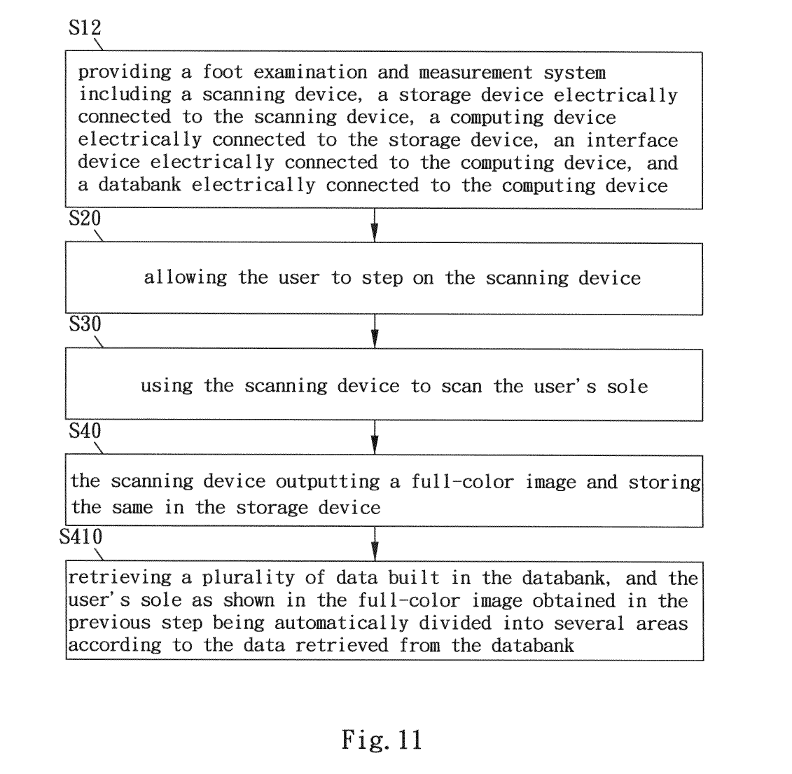

In a foot examination and measurement system according to a second embodiment of the present invention, a databank 50 is further included, as shown in FIG. 1B, for assisting in dividing a user's sole into different areas. FIG. 11 is a flowchart showing the steps included in the method for foot examination and measurement according to the present invention for dividing a user's sole into different areas. In a first step S12, the foot examination and measurement system including the databank 50 according to the second embodiment of the present invention is prepared. In a second step S20, the user steps on the scanning device 10. In a third step S30, use the scanning device 10 to scan the user's sole. In a fourth step S40, the scanning device 10 outputs a full-color image and stores the same in the storage device 20. In a fifth step S410, a plurality of data built in the data bank 50 is retrieved, and the user's sole as shown in the full-color image obtained in the step S40 is automatically divided into several areas according to the data retrieved from the databank 50.

In an embodiment of the present invention, the method for dividing the user's sole into different areas further includes a manual measurement step, in which the user adjusts the positions and sizes of the areas defined on the user's sole through the above steps.

With the above-described method, the foot examination and measurement system of the present invention is able to automatically divide the user's sole into several areas and thereby enables a non-professional person to help the user in choosing a shoe shape that is most suitable for the user. On the other hand, the foot examination and measurement method of the present invention includes the manual measurement step for a professional person to more accurately divide the user's sole into different areas.

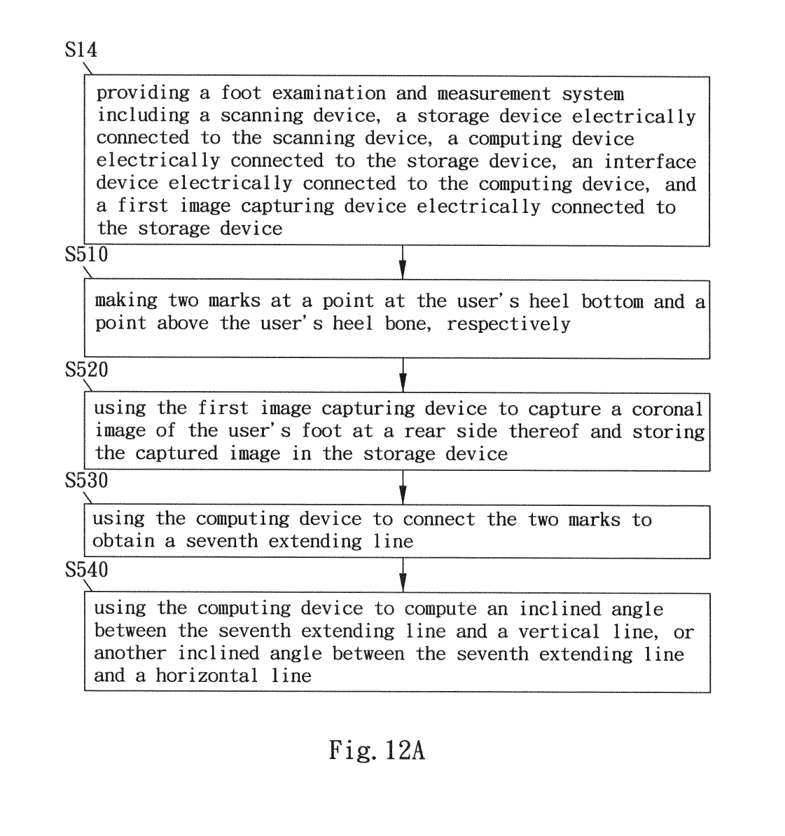

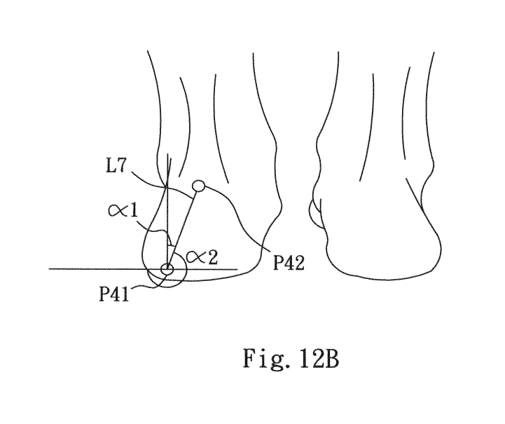

In a foot examination and measurement system according to a third embodiment of the present invention, a first image capturing device 60 is further included, as shown in FIG. 1C, for measuring a user's foot angle. FIG. 12A is a flowchart showing the steps included in the method for foot examination and measurement according to the present invention for measuring a user's foot angle; and FIG. 12B is a conceptual view showing the measurement of a user's foot angle according to the method of the present invention. In a first step S14, the foot examination and measurement system including the first image capturing device 60 according to the third embodiment of the present invention is prepared. In a second step S510, two marks P41 and P42 are attached to a point at the user's heel bottom and a point above the user's heel bone, respectively. In a third step S520, the first image capturing device 60 is used to capture a coronal image of the user's foot at a rear side thereof and the captured image is stored in the storage device 20. In a fourth step S530, the computing device 30 connects the mark P41 with the mark P42 to obtain a seventh extending line L7. In a fifth step S540, the computing device 30 computes an inclined angle α₁ between the seventh extending line L7 and a vertical line, or an inclined angle α₂ between the seventh extending line L7 and a horizontal line, as shown in FIG. 12B.

With the above-described method, the foot examination and measurement system of the present invention can be used to measure a user's foot angle, and thereby help the user in choosing a suitable insole.

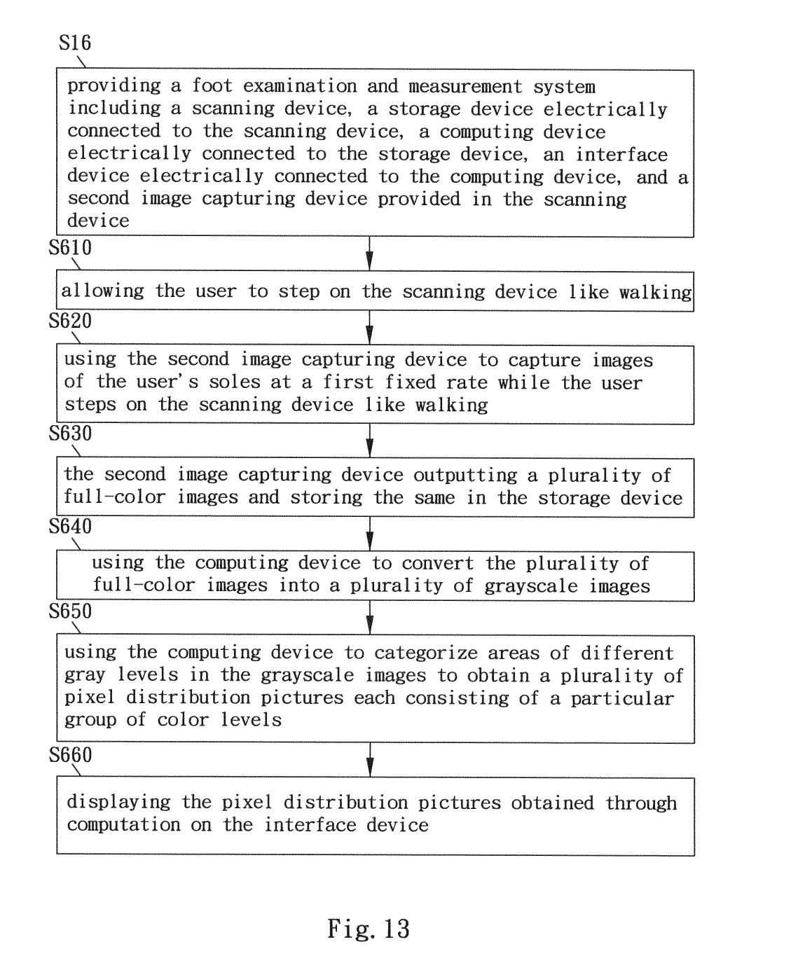

In a foot examination and measurement system according to a fourth embodiment of the present invention, a second image capturing device 70 is further included, as shown in FIG. 1D, for measuring a user's foot pressure when the user steps on the scanning device like walking. FIG. 13 is a flowchart showing the steps included in the method for foot examination and measurement according to the present invention for measuring a user's foot pressure distribution when the user steps on a scanning device like walking. As shown in FIG. 13, in a first step S16, the foot examination and measurement system including the second image capturing device 70 according to the fourth embodiment of the present invention is prepared. In a second step S610, allow the user to step on the scanning device 10 like walking. In a third step S620, while the user steps on the scanning device 10 like walking, the second image capturing device 70 is used to capture images of the user's soles at a first fixed rate. In a fourth step S630, the second image capturing device 70 outputs a plurality of full-color images, which are then stored in the storage device 20. In a fifth step S640, the computing device 30 converts the plurality of full-color images into a plurality of grayscale images In a sixth step S650, the computing device 30 categorizes areas of different gray levels in the grayscale images to obtain a plurality of pixel distribution pictures each consisting of a particular group of color levels. In a seventh step S660, the pixel distribution pictures obtained through computation are displayed on the interface device 40.

In an embodiment of the present invention, the method for measuring the user's foot pressure distribution when the user steps on the scanning device like walking further includes a dynamic playback step. In this step, the plurality of pixel distribution pictures is continuously played back at a second fixed rate. For example, the first and the second fixed rate can be 15 frames per second.

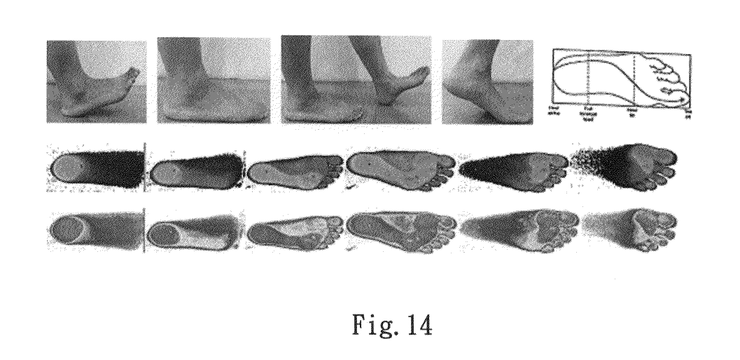

Please also refer to FIG. 14 that shows full-color scanned images that are obtained using the foot examination and measurement method of the present invention when the user steps on a scanning device like walking and show changes in a user's foot pressure distribution.

Therefore, the present invention can provide a simple foot examination and measurement technique, in which the general image conversion principle is used to convert simple scanned pictures and images into foot pressure distribution pictures without the need of using special and expensive platform or hardware. With the advantages thereof, the foot examination and measurement system and method according to the present invention can be easily promoted and become popularized in foot examination and shoe market, allowing more non-professional people to engage in the shoe market and therefore enabling quick upgrade of the shoe industry.

From the above descriptions, it can be found the present invention is novel, improved, and industrially practical for use. More specifically, the foot examination and measurement system of the present invention is novel and improved because it includes a scanning device, a storage device, a computing device, and an interface device to achieve the function of examining and measuring a user's foot to obtain different information about the user's foot. Moreover, the foot examination and measurement system of the present invention can further include a database for measuring different areas of the user's sole, a first image capturing device for measuring the user's heel angle, and a second image capturing device for measuring changes in the user's foot pressure distribution when the user walks. Therefore, the present invention is largely helpful in assisting the user to choose suitable shoes and/or insoles, and improving the user's foot and whole body health. Accordingly, it is predictable the present invention and products derived therefrom would fully meet the current market demands.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A foot examination and measurement system, comprising:
a scanning device being used to scan a user's foot when the user steps on the scanning device;
a storage device being electrically connected to the scanning device for storing information about the user's foot outputted by the scanning device;
a computing device being electrically connected to the storage device for computing the information about the user's foot; and
an interface device being electrically connected to the computing device for displaying the user's foot information and computation results from the computing device.

2. The foot examination and measurement system as claimed in claim 1, further comprising a databank electrically connected to the computing device for storing a plurality of data about foot shapes for different users.

3. The foot examination and measurement system as claimed in claim 1, further comprising a first image capturing device electrically connected to the storage device for capturing images of a user's foot.

4. The foot examination and measurement system as claimed in claim 1, further comprising a second image capturing device provided in the scanning device for capturing dynamic images of a user's feet when the user steps on the scanning device like walking.

5. A foot examination and measurement method for examining and measuring a user's foot pressure distribution, comprising the following steps:
providing a foot examination and measurement system including a scanning device, a storage device electrically connected to the scanning device, a computing device electrically connected to the storage device, and an interface device electrically connected to the computing device;
allowing the user to step on the scanning device;
using the scanning device to scan the user's sole;
the scanning device outputting a full-color image and storing the same in the storage device;
using the computing device to convert the full-color image into a grayscale image;
using the computing device to categorize areas of different gray levels in the grayscale image to obtain a pixel distribution picture consisting of a particular group of color levels; and
displaying the pixel distribution picture obtained through computation on the interface device.

6. The foot examination and measurement method as claimed in claim 5, wherein, after the step of using the computing device to convert the full-color image into a grayscale image, a further step is included to remove a background from the full-color image.

7. The foot examination and measurement method as claimed in claim 5, wherein, in the step of using the computing device to convert the full-color image into a grayscale image, a conversion formula is used to convert the full-color image into the grayscale image.

8. The foot examination and measurement method as claimed in claim 7, wherein the conversion formula is:
Y=(9798×r+19235×g+3735×b)/32768
where, y is a gray value of each gray level, and r, g, and bare corresponding intensities of red, green, and blue colors, respectively.

9. The foot examination and measurement method as claimed in claim 8, wherein, in the step of using the computing device to categorize areas of different gray levels in the grayscale image to obtain a pixel distribution picture consisting of a particular group of color levels, every specific range of the gray values is defined as one color level, and the gray levels in the grayscale image are sequentially converted into their respectively correspondent specific color level according to their respective gray value.

10. The foot examination and measurement method as claimed in claim 8, further comprising the following steps to convert a two-dimensional full-color image into a three-dimensional image:
using the computing device to convert every gray level into a height value;
using the computing device to combine the full-color image with the height values and compute to obtain a three-dimensional image; and
displaying the three-dimensional image on the interface device.

11. The foot examination and measurement method as claimed in claim 8, further comprising a step of using the computing device to calculate the user's foot-ground contact area by dividing a sum of all adjoining pixel blocks having a gray value above 144 by a total number of the pixels in the grayscale image.

12. A foot examination and measurement method for measuring a user's actual foot length, comprising the following steps:
providing a foot examination and measurement system including a scanning device, a storage device electrically connected to the scanning device, a computing device electrically connected to the storage device, and an interface device electrically connected to the computing device;
allowing the user to step on the scanning device;
using the scanning device to scan the user's sole;
the scanning device outputting a full-color image and storing the same in the storage device;
using the computing device to mark a highest point on the full-color image at a position corresponding to a most front point on the user's second toe;
using the computing device to locate a lowest point on the full-color image from bottom to top; and
using the computing device to locate a centerline of the user's sole by connecting the highest point and the lowest point, calculate a distance between the highest and the lowest point to determine an actual length of the user's foot.

13. The foot examination and measurement method for measuring a user's actual foot length as claimed in claim 12, further comprising the following steps for measuring a width of a user's forefoot:
using the computing device to locate a near-front point on the centerline with a distance between the near-front point and the highest point of the centerline being about 34% of a full length of the centerline, and then extend a line from the near-front point toward two points on the full-color image that are corresponding to two prominent points on an inner and an outer side of the user's forefoot, so as to obtain a forefoot cross line extended between the two prominent points; and
using the computing device to calculate a distance of the forefoot cross line.

14. The foot examination and measurement method for measuring a user's actual foot length as claimed in claim 13, further comprising a manual measurement step of allowing the user to adjust positions of the two prominent points.

15. The foot examination and measurement method for measuring a user's actual foot length as claimed in claim 12, further comprising the following steps for measuring a width of a user's middle foot:
using the computing device to locate a near-rear point on the centerline with a distance between the near-rear point and the lowest point of the centerline being about 33% of a full length of the centerline, and extend a line from the near-rear point toward two points on the full-color image that are corresponding to two innermost points on an inner and an outer side of the user's middle foot to obtain a middle-foot cross line extended between the two innermost points; and
using the computing device to calculate a distance of the middle-foot cross line.

16. The foot examination and measurement method for measuring a user's actual foot length as claimed in claim 15, further comprising a manual measurement step of allowing the user to adjust positions of the two innermost points.

17. The foot examination and measurement method for measuring a user's actual foot length as claimed in claim 12, further comprising the following steps for measuring a user's foot eversion angle:
using the computing device to locate a first axis point on the centerline with a distance between the first axis point and the lowest point of the centerline being about 29% of a full length of the centerline;
using the computing device to locate a second axis point on the centerline with a distance between the second axis point and the highest point of the centerline being about 38% of a full length of the centerline;
using the computing device to extend a first extending line from the second axis point perpendicular to the centerline, so that the first extending line intersects with an outline of the full-color image to produce two contact points;
using the computing device to compute a distance between the two contact points, and locate a middle point between the two contact point to serve as a third axis point;
using the computing device to locate a second extending line extended between the first axis point and the third axis point;
using the computing device to compute an eversion angle between the centerline and the second extending line.

18. The foot examination and measurement method for measuring a user's actual foot length as claimed in claim 12, further comprising the following steps for dividing a user's sole into different areas:
Providing a databank electrically connected to the computing device for storing a plurality of data about foot shapes for different users; and
retrieving a plurality of data built in the databank, and the user's sole as shown in the full-color image obtained in the previous step being automatically divided into several areas according to the data retrieved from the databank.

19. The foot examination and measurement method for dividing a user's sole into different areas as claimed in claim 18, further comprising a manual measurement step of allowing the user to adjust positions and sizes of the areas defined on the user's sole.

20. A foot examination and measurement method for measuring an inclined angle of a user's first and fifth toe, comprising the following steps:
providing a foot examination and measurement system including a scanning device, a storage device electrically connected to the scanning device, a computing device electrically connected to the storage device, and an interface device electrically connected to the computing device;
allowing the user to step on the scanning device;
using the scanning device to scan the user's sole;
the scanning device outputting a full-color image and storing the same in the storage device;
using the computing device to compute and locate in the full-color image two points that correspond to two prominent points on an inner side of the user's heel bone and forefoot;
using the computing device to connect the two prominent points on the inner side of the user's heel bone and forefoot to obtain a third extending line;
using the computing device to inwardly rotate the third extending line about the prominent point on the inner side of the user's forefoot by an angle so that the third extending line touches the full-color image and thereby produces a first contacting point;
using the computing device to connect the prominent point on the inner side of the user's forefoot and the first contacting point to obtain a fourth extending line; and
using the computing device to calculate an angle between the third extending line and the fourth extending line to obtaining the inclined angle of the user's first toe;
using the computing device to compute and locate in the full-color image two points that correspond to two prominent points on an outer side of the user's heel bone and forefoot;
using the computing device to connect the two prominent points on the outer side of the user's heel bone and forefoot to obtain a fifth extending line;
using the computing device to inwardly rotate the fifth extending line about the prominent point on the outer side of the user's forefoot by an angle so that the fifth extending line touches the full-color image and thereby produces a second contacting point;
using the computing device to connect the prominent point on the outer side of the user's forefoot and the second contacting point to obtain a sixth extending line; and
using the computing device to calculate an angle contained between the fifth extending line and the sixth extending line to obtaining the inclined angle of the user's fifth toe.

21. A foot examination and measurement method for measuring a user's foot pressure distribution when the user steps on a scanning device like walking, comprising the following steps:
providing a foot examination and measurement system including a scanning device, a storage device electrically connected to the scanning device, a computing device electrically connected to the storage device, an interface device electrically connected to the computing device, and a second image capturing device provided in the scanning device;
allowing the user to step on the scanning device like walking;
using the second image capturing device to capture images of the user's soles at a first fixed rate while the user steps on the scanning device like walking;
the second image capturing device outputting a plurality of full-color images and storing the same in the storage device;
using the computing device to convert the plurality of full-color images into a plurality of grayscale images;
using the computing device to categorize areas of different gray levels in the grayscale images to obtain a plurality of pixel distribution pictures each consisting of a particular group of color levels; and
displaying the pixel distribution pictures obtained through computation on the interface device.

22. The foot examination and measurement method as claimed in claim 21, further comprising a dynamic playback step of continuously playing back the plurality of pixel distribution pictures at a second fixed rate.
